(19) United States
(12) Patent Application Publication
(10) Pub. No.: US 2010/0069504 A1
(43) Pub. Date: Mar. 18, 2010

(54) AMINOBENZOCYCLOHEPTENE DERIVATIVES, METHODS FOR PREPARING THE SAME AND USES THEREOF IN THERAPY

(75) Inventors: Céline Tarnus-Rondeau, Rixheim (FR); Albert Defoin, Mulhouse (FR); Sébastien Albrecht, Mulhouse (FR); Anamaria Maiereanu, Strasbourg (FR); Nadège Faux, Schiltigheim (FR); Patrick Pale, Strasbourg (FR)

Correspondence Address: YOUNG & THOMPSON, 209 Madison Street, Suite 500, Alexandria, VA, 22314, US

(73) Assignees: UNIVERSITE DE HAUTE ALSACE, Mulhouse Cedex (FR); UNIVERSITE LOUIS PASTEUR, Strasbourg Cedex (FR)

(21) Appl. No.: 12/513,262

(22) PCT Filed: Oct. 31, 2007
(86) PCT No.: PCT/FR07/01812

§ 371 Date: May 1, 2009

(30) Foreign Application Priority Data
Nov. 3, 2006 (FR) 06/09615

(51) Int. Cl. A61K 31/135; C07C 211/33; A61P 35/00

(52) U.S. Cl. 514/657; 564/428; 564/427; 514/656

(57) ABSTRACT

A compound of the general formula (I) as an active principle for treating cancer, specifically tumors, in particular diseases involving inhibition of metalloproteases, such as Aminopeptidase-N. Pharmaceutical compositions comprising the compound of general formula (I). Methods of treatment using the compound of general formula (I).

The present invention relates to aminobenzocycloheptene derivatives, methods for preparing the same, and uses thereof in therapy.

More particularly, the present invention relates to new aminobenzocycloheptene derivative compounds for use in therapy, in phenomena relating to oncology and more particularly in the angiogenesis process.

Angiogenesis is a process currently widely investigated in the field of cancer research. Studies (R. T. Poon et al, J. Cli. Oncol., 2001, 19, 1207-1225) more particularly show that the neoangiogenesis process is essential to the tumour development. Neutralizing this process in the case of cancers will therefore be a promising approach to develop new anticancer treatments enabling to control the progress of the tumour, and which are less toxic than those used up to now.

It has recently been shown that aminopeptitade N, hereinafter called AP-N, or CD13, is involved in cellular mobility phenomena. It is thus considered as an important regulator of endothelial morphogenesis during the angiogenesis process (H. Hashida et al, Gastroenterology, 2002, 122, 376-386; S. W. Bhagwat et al, Blood, 2001, 97, 652-659). Studies (R. Pasqualini et al, Cancer Res., 2000, 60, 722-727) show that the antibodies directed against AP-N and its catalyst activity or low molecular weight inhibitors such as bestatine or actinonine have a negative effect on tumour growth in mouse models. However, bestatine, actinonine or amastatine are low selectivity inhibiting molecules. Indeed, given that there is a great number of aminopeptidases that are structurally very close to each other and that act according to very similar catalytic mechanisms, it is very difficult to design selective inhibitors.

A new more powerful and more selective series of AP-N inhibiting molecules has been discovered, that is 3-amino-2-tetralone and derivatives thereof (C. Schalk et al, Arch. Biochem. Biophys., 1994, 311(1), 42-46).

Even though it has a great selectivity, this molecule has the drawback not to be very stable in aqueous solution.

Therefore, there is a need to provide new more powerful, more selective and chemically stable AP-N inhibiting molecules.

For this purpose, and in accordance with the present invention, there is provided new aminobenzocycloheptene derivative compounds having the general formula (I):

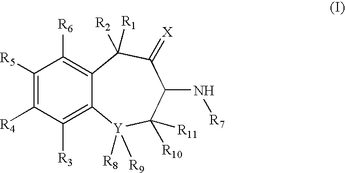

wherein,

R₁ represents an hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical, a (C₁-C₆)alkoxy radical, a (C₁-C₆)aralkyloxy radical, a (C₁-C₆)alkylthio radical, a (C₁-C₆) aralkylthio radical;

R₂ represents an hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆) heteroaralkyl radical; where R₁ and R₂ can form together an unsubstituted or substituted carbon ring or an unsubstituted or substituted heterocycle; or R₁ can be bonded to the heptene ring through a double bond, R₂ being then absent;

R₃, R₄, R₅ and R₆, the same or different, represent independently of each other a hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a polyfluoro (C₁-C₆) alkyl radical, a (C₁-C₆)aralkyl radical, a (C₁-C₆)heteroaralkyl radical, a (C₁-C₆)alkoxy radical, an aryl or heteroaryl group; R₃ and R₄, R₄ and R₅, R₅ and R₆ independently of each other can form together a methylenedioxy radical joining the adjacent carbon atoms or an unsubstituted or substituted aromatic carbon ring or an unsubstituted or substituted aromatic heterocycle;

R₇ represents a hydrogen atom, a (C₁-C₆)alkyl radical;

X is an oxygen atom, a sulphur atom, an imine radical N—R₁₂, an oxime radical N—O—R₁₃, wherein R₁₂ and R₁₃ represent a hydrogen atom, a (C₁-C₆)alkyl radical, a (C₁-C₆)(cycloalkyl)alkyl radical, a (C₁-C₆) (heterocycloalkyl)alkyl radical, a (C₁-C₆)aralkyl radical, a (C₁-C₆)heteroaralkyl radical;

Y is a carbon atom; a nitrogen atom, R₈ or R₉ being then absent; an oxygen atom, a sulphur atom, R₈ and R₉ being then absent;

R₈ and R₁₀, the same or different, represent independently of each other a hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical, a (C₁-C₆)alkoxy radical, a (C₁-C₆)aralkyloxy radical, a (C₁-C₆)alkylthio radical, a (C₁-C₆)aralkylthio radical;

R₉ and R₁₁, the same or different, represent independently of each other a hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical, a (C₁-C₆)alkoxy radical, a (C₁-C₆)aralkyloxy radical, a (C₁-C₆)alkylthio radical, a (C₁-C₆)aralkylthio radical, where R₉ and R₁₁ can form together an unsubstituted or substituted carbon ring or an unsubstituted or substituted heterocycle or form a double bond with the two adjacent carbon atoms of the heptene ring;

optical and geometrical isomers thereof in particular enantiomeric ordiastomeric forms and mixtures thereof, in particular racemic mixtures, as well as inorganic and organic acid addition salts thereof, except for the compound wherein R₄, R₅ and R₆ represent a methoxy radical, R₁, R₂, R₃, R₇, R₈, R₉, R₁₀, R₁₁ represent a hydrogen atom, X represents an oxygen atom and Y represents a carbon atom.

These acids are advantageously pharmaceutically acceptable acids, even though other acids can be used. The acids are, for example, hydrochloric, hydrobromic, hydriodic, nitric, sulphuric, phosphoric, acetic, mono- or bi- or tri-haloacetic, formic, benzoic, maleic, fumaric, succinic, tartaric, citric, oxalic, glyoxylic, aspartic, alkanesulphonic, benzenesulphonic or toluenesulphonic acids.

In the present description, the alkyl, (cycloalkyl)alkyl, (heterocycloalkyl)alkyl, aralkyl, heteroaralkyl, alkoxy, aralkyloxy, alkylthio, aralkylthio radicals are straight or branched.

For the (C₁-C₆) (cycloalkyl)alkyl or (C₁-C₆)aralkyl, (C₁-C₆) indicates the number of carbon atoms in the alkyl portion.

A group of preferred compounds according to the invention corresponds to compounds wherein R₁ represents a hydrogen atom, a fluorine atom, a (CH₂)ₙPh radical, a S(CH₂)ₙPh radical, n ranging from 1 to 6, and preferably from 1 to 5.

Another group of preferred compounds according to the invention corresponds to compounds wherein R₂ is a hydrogen atom.

Another group of preferred compounds according to the invention corresponds to compounds wherein X is an oxygen atom.

Another group of preferred compounds according to the invention corresponds to compounds wherein Y is a carbon atom.

Another group of preferred compounds according to the invention corresponds to compounds wherein R₃, R₄, R₅ and R₆ the same or different, represent independently of each other a hydrogen atom, a bromine atom, a phenyl radical, or R₃ and R₄, R₄ and R₅, R₅ and R₆ independently of each other form together an unsubstituted or substituted aromatic carbon ring.

Another group of preferred compounds according to the invention corresponds to compounds wherein R₇ is a hydrogen atom.

Another group of preferred compounds according to the invention corresponds to compounds wherein simultaneously Y is a carbon atom and R₈, R₉, R₁₀, and R₁₁ are hydrogen atoms.

Particularly preferred compounds are those wherein R₂ and R₇ are simultaneously a hydrogen atom, X is an oxygen atom, and Y is a carbon atom.

Among these particularly preferred compounds, a group of most particularly preferred compounds corresponds to compounds wherein R₁ is a hydrogen atom, a fluorine atom, a benzylthio radical, a (CH₂)ₙPh radical, where n=1-5.

Among these particularly preferred compounds, another group of most particularly preferred compounds corresponds to compounds wherein R₃, R₄, R₅, R₆ are simultaneously a hydrogen atom.

Among these particularly preferred compounds, another group of most particularly preferred compounds corresponds to compounds wherein R₄ and R₅ are a hydrogen atom, and R₃ and R₆ represent independently of each other a hydrogen atom, a bromine atom, a phenyl radical, with the proviso that R₃ and R₆ are not simultaneously a hydrogen atom.

Among these particularly preferred compounds, another group of most particularly preferred compounds corresponds to compounds wherein R₃ and R₆ are a hydrogen atom, and R₄ and R₅ represent independently of each other a hydrogen atom, a bromine atom, a phenyl radical, with the proviso that R₄ and R₅ are not simultaneously a hydrogen atom.

Among these particularly preferred compounds, another group of most particularly preferred compounds corresponds to compounds wherein R₃ and R₄, R₅ and R₆ independently of each other, form together an unsubstituted or substituted aromatic carbon ring, joining the adjacent carbon atoms.

Particularly preferred compounds according to the invention have the formulae (Ia) to (Ie) below:

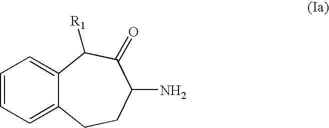

wherein R₁ represents a hydrogen atom, a fluorine atom, the CH₂Ph radical, the (CH₂)₂Ph radical, the (CH₂)₃Ph radical, the (CH₂)₄Ph radical, the (CH₂)₅Ph radical, the S—CH₂Ph radical, the ═CH-Ph radical;

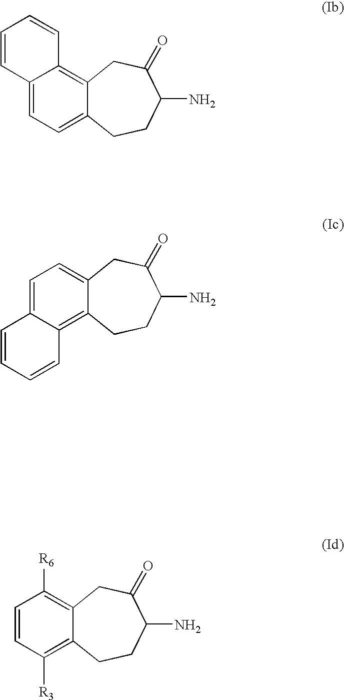

wherein the substituents R₃ and R₆ are as defined in the following tables I and II:

TABLE I

| R₃ | R₆ |
|---|---|
| H | phenyl |
| H | Br |
| phenyl | H |
| Br | H |
| phenyl | Br |
| Br | phenyl |
| phenyl | phenyl |
| Br | Br |

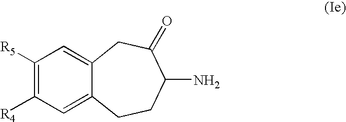

TABLE II

| R₄ | R₅ |
|---|---|
| H | phenyl |
| H | Br |
| phenyl | H |
| Br | H |
| phenyl | phenyl |
| Br | Br |

Of course, all optical and geometrical isomers, in particular the enantiomeric or diastereomeric forms and mixtures thereof, in particular racemic mixtures, as well as inorganic and organic acid addition salts of the above described compounds belong to the present invention.

The present invention also relates to a method for preparing compounds of the formula (I) wherein R₁ represents a hydrogen atom, a fluorine atom, a (CH₂)ₙPh radical, the ═CH-Ph radical, R₂ is a hydrogen atom or is absent, R₇, R₈, R₉, R₁₀, and R₁₁ are hydrogen atoms; X is an oxygen atom, a NOH radical, Y is a carbon atom, R₃, R₄, R₅, R₆ have the meanings already set out; and salts thereof, wherein

1) a —NHPG protected amine function is introduced onto the compound of the general formula (II)

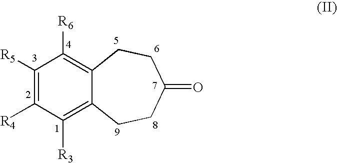

at the 7-position by reacting the ketone function, wherein PG is a protecting group, and at the 6-position, a ketone function is introduced when X is an oxygen atom or a ketone-oxime function is introduced when X is the NOH radical to form a derivative of the general formula (III)

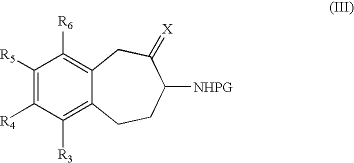

2) when R₁ is not a hydrogen atom, the function corresponding to R₁ is introduced at the 5-position to form a derivative of the general formula (IV)

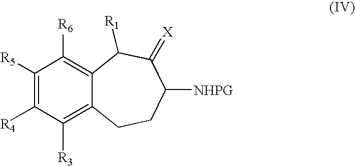

3) the amine NH-PG function is deprotected by cleaving the PG group.

The amino function at the 7-position from the ketone function can be obtained by condensation reaction of the ketone function with a primary amine followed by a reduction, for example with NaBH₄.

In the scope of the present invention, by protecting group PG is meant a group that enables on the one hand to protect the amine reactive function during the synthesis of compounds and on the other hand to regenerate this reactive function intact at the end of synthesis. Such a protecting group is for example, N-tert-butoxycarbonyl(Boc), with the deprotection being carried out by acid hydrolysis in the presence of, for example, hydrochloric acid.

The ketone function at the 6-position when X is an oxygen atom can be obtained by introduction of a hydroxyl group at the 6-position followed by an oxidation with, for example, Dess-Martin periodinane.

The present invention also relates to a method for preparing compounds of the formula (I) wherein R₁ represents a S(CH₂)ₙPh radical, R₂ is a hydrogen atom, R₇, R₈, R₉, R₁₀, and R₁₁ are hydrogen atoms; X is an oxygen atom, Y is a carbon atom, R₃, R₄, R₅, R₆ have the meanings already set out; and salts thereof, wherein

1) a double bond is formed onto the compound of the general formula (II)

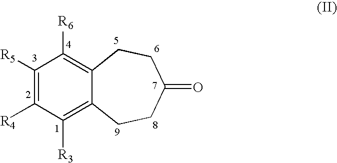

between the 5- and 6-positions

2) a —NHPG protected amine function is introduced at the 7-position by reacting the ketone function, wherein PG is a protecting group to form a derivative of the general formula (V)

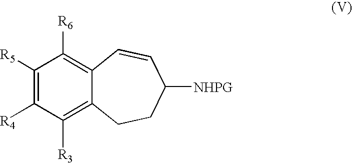

3) the double bond is oxidized to form an epoxide function bonding together the carbon atoms at the 5- and 6-positions

4) the S(CH₂)ₙPh radical is introduced at the 5-position to form a derivative of the general formula (VI)

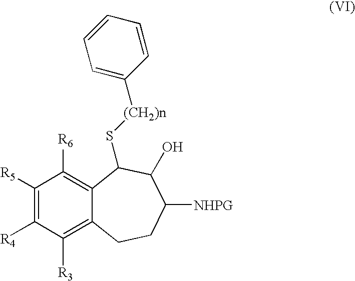

5) the alcohol function of the resulting derivative is oxidized

6) the NH-PG amine function is deprotected by cleaving the PG group.

The operating conditions used in the different above described steps are conventional for those skilled in the art.

The compound of the general formula (II) is described in the literature where:
R₆, R₄, R₃ represent a hydrogen atom and R₅ represents a CF₃, CH₃, C(CH₃)₃, or OCH₃ radical
R₅ and R₄ represent a hydrogen atom and R₆ and R₃ represent a OCH₃ radical
R₅ and R₄ represent a hydrogen atom and R₆ and R₃ represent a CH₃ radical
R₅ and R₄ represent a bromine atom and R₆ and R₃ represent a OCH₃ radical
R₆ and R₃ represent a hydrogen atom and R₅ and R₄ represent a OCH₃ radical.

As will be seen in the examples hereinafter, the compounds according to the present invention are more selective AP-N inhibitors than inhibitors known up to now, with an inhibition constant Ki lower than 10⁻⁵M and higher than 10⁻³M with other aminopeptidases.

The present invention also relates to a pharmaceutical composition, containing as an active principle, a compound of the formula (I) such as described above, or pharmaceutically acceptable inorganic and organic acid addition salts thereof, with the proviso that the compound wherein R₄, R₅ and R₆ represent a methoxy radical, R₁, R₂, R₃, R₇, R₉, R₉, R₁₀, R₁₁ represent a hydrogen atom, X represents an oxygen atom and Y represents a carbon atom, is not excluded. These compositions comprise an effective dose of one of these compounds, or a pharmaceutically acceptable salt thereof, and the active principle can optionally be mixed with at least a pharmaceutically acceptable excipient. These excipients are known to those skilled in the art and are adapted for the pharmaceutical form and to the administration mode desired.

The pharmaceutical compositions according to the invention have all the forms known to those skilled in the art, in particular adapted for an oral, sublingual, intramuscular, intravenous, topical, local, intranasal, transdermal or rectal administration. The pharmaceutical compositions can thus have the form of gelatine capsules, tablets, granules, suppositories, injectable preparations, creams, prepared according to common methods.

The invention also relates to the use of a compound of the formula (I) according to the invention, with the proviso that the compound wherein R₄, R₅ and R₆ represent a methoxy radical, R₁, R₂, R₃, R₇, R₈, R₉, R₁₀ and R₁₁ represent a hydrogen atom, X represents an oxygen atom and Y represents a carbon atom is not excluded, for making a drug for treating cancers, tumours, and in particular for treating and preventing diseases involving inhibition of metalloproteases, more particularly the aminopeptidase-N.

The following examples will illustrate the present invention, yet without limiting the scope thereof.

### PREPARATIONS

### Preparation 1: 1-bromo-2,3-bis-bromomethyl-benzene

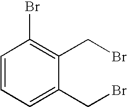

A 3-bromo-o-xylene (2 g, 10.8 mmoles) and N-bromosuccinimide (4.04 g, 22.7 mmoles) solution is irradiated in a carbon tetrachloride (70 mL) with a HPK125 mercury lamp for 2 hours. The reaction mixture is diluted with ethyl acetate, and then washed in aqueous ammonium chloride (2M) and dried on magnesium sulphate. The solvent is vaporized off to obtain 3.7 g of a colourless oil.

¹HNMR (CDCl₃): 7.56 (d, J=8.1 Hz, 1 Har); 7.31 (d, J=7.5 Hz, 1 Har); 7.15 (t, J=7.8 Hz, 1 Har); 4.84 (s, CH Br); 4.64 (s, CH₂Br).

This compound is used instead of α-α′-dibromo-o-xylene used in preparation 2 when a compound having a bromine atom as a substituent R₃ or R₆ is desired to be synthesized.

### Preparation 2: dimethyl 7-oxo-5,6,8,9-tetrahydrobenzocycloheptene-6,8-dicarboxylate

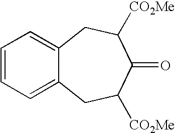

A α-α′-dibromo-o-xylene (51 g, 189 mmoles), dimethyl 1,3-acetonedicarboxylate (49.3 g, 283 mmoles), tetrabutylammonium bromide (38.3 g, 118.8 mL) mixture is heated in an 1N aqueous sodium hydrogencarbonate solution (1 L) and dichloromethane (400 mL) at 40° C. and under argon overnight under a vigorous stirring. The organic phase is separated and evaporated to dryness. The residue is diluted with ethyl acetate, washed in brine (4×100 mL), and then dried on magnesium sulphate. A yellow resin (80 g) is obtained and used without being further purified.

Melting point=100-110° C.

Major trans isomer, ¹HNMR (CDCl₃): 7.23 (m, 4 Har); 3.93 (dd, H—C(6), H—C(8)); 3.70 (s, COOMe); 3.23 (dd, Ha-C(5), Ha-C(9)); 3.14 (m, Hb-C(5), Hb-C(9)); J(5a,5b)=15.0 Hz, J(5a,6)=9.0 Hz, J(5b,6)=3.5 Hz.

Minor cis isomer, ¹HNMR (CDCl₃): 7.26 (m, 4 Har); 3.79 (s, COOMe); 3.56 (dd, H—C(6), H—C(8)); 3.22 (m, Ha-C(5), Ha-C(9)); 3.15 (m, Hb-C(5), Hb-C(9)); J(5a, 5b)=14.8 Hz, J(5a, 6)=11.3 Hz, J(5b, 6)=3.3 Hz.

### Preparation 3: 5,6,8,9-tetrahydro-benzocyclohepten-7-one (compound having the formula II)

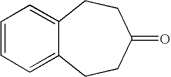

The isomeric mixture of dimethyl 7-oxo-5,6,8,9-tetrahydrobenzocycloheptene-6,8-dicarboxylate (80 g) obtained according to preparation 2 is refluxed in a 3M aqueous sulphuric acid (300 mL) and acetonitrile (50 mL) solution overnight under argon. The mixture is diluted with diethyl ether, neutralized with a 2M aqueous sodium hydroxide solution (3×300 mL), dried on magnesium sulphate and evaporated to dryness. The residue is distilled off at 97-98° C. under 0.4-0.5 Torr to obtain colourless crystals (26.1 g, 84% from α,α′-dibromo-o-xylene).

Melting point: 43-44° C.

¹HNMR (CDCl₃): 7.23 (m, 4 Har); 2.91 (m, 2H—C(6), 2H—C(8)); 2.62 (m, 2H—C(5), 2H—C(9)).

### Preparation 4: 7-(tert-butoxycarbonyl-amino)-6,7,8,9-tetrahydro-5H-benzocyclohepten-6-ol

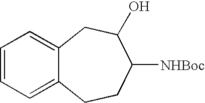

Boc=tert-butoxycarbonyl

To a solution of 5,6,8,9-tetrahydro-benzocyclohepten-7-one (1.07 g, 6.68 mmoles) (preparation 3), triethylamine (1.3 mL, 9.35 mmoles), in anhydrous toluene (15 mL), trimethylsilyl trifluoromethanesulphonate (1.45 mL, 8.01 mmoles) is added dropwise at room temperature under argon. The reaction mixture is heated at 90° C. for 2 hours, diluted with cyclohexane, washed with a 2M aqueous ammonium chloride solution and brine. The organic phase is dried on magnesium sulphate and evaporated to obtain silylenol ether which is used without being further purified.

To a mixture of silylenol ether (6.68 mmoles) in anhydrous dichloromethane (20 mL), 3-chloroperoxybenzoic acid (1.4 g, 8.01 mmoles) is added portionwise at 0° C. under argon. The reaction mixture is stirred at 0° C. for 2 hours, the 3-chlorobenzoic acid precipitate is filtered and the filtrate is evaporated to obtain the hydroxy-ketone which is used without being further purified.

A mixture of hydroxy-ketone (6.68 mmoles), titanium isopropoxide (IV) (4 mL, 13.3 mmoles) and saturated ammonia in ethanol (20 mL) is stirred under argon at room temperature overnight. Then, sodium borohydride (380 mg, 10 mmoles) is added and the resulting mixture is stirred at room temperature for 2 hours. The solvents are evaporated off, the residue is diluted with ethyl acetate and 1N aqueous ammonium hydroxide (20 mL) is added. The resulting inorganic precipitate is filtered, washed with a 1/1 ethyl acetate/1N aqueous ammonium hydroxide mixture (3×20 mL). The organic phase is separated and the remaining aqueous phase is extracted with ethyl acetate (3×20 mL). The combined organic extracts are dried on magnesium sulphate, and concentrated to dryness to obtain raw amino alcohol.

A mixture of this amino-alcohol (6.68 mmoles), di-tert-butyl dicarbonate (3.2 g, 14.6 mmoles) and sodium carbonate (780 mg, 7.34 mmoles) in methanol (10 mL) is stirred under argon overnight. The solvent is evaporated off, the resulting solid is washed with water (3×20 mL) and cold isopropyl ether (three times) to obtain 7-(tert-butoxycarbonyl-amino)-6,7,8,9-tetrahydro-5H-benzocyclohepten-6-ol as colourless crystals (985 mg, 53% from 5,6,8,9-tetrahydro-benzocyclohepten-7-one).

Melting point: 178-180° C.

### Preparation 5: 7-(benzyloxycarbonyl-amino)-6,7,8,9-tetrahydro-5H-benzocyclohepten-6-ol

The synthesis described according to preparation 4 is repeated using 3 g (18.7 mmoles) of 5,6,8,9-tetrahydro-benzocyclohepten-7-one (preparation 3).

When the amino-alcohol is obtained, a mixture of this amino-alcohol (18.7 mmoles), benzyl chloroformate (3.8 mL, 26.2 mmoles) in THF (40 mL) is stirred with sodium carbonate (5.6 g, 52.4 mmoles) under argon overnight at room temperature. The reaction mixture is diluted with ethyl acetate and washed with 2M aqueous ammonium chloride, brine, and then dried on magnesium sulphate. The solvent is evaporated off, the solid obtained is washed with isopropyl ether to obtain 7-(benzyloxycarbonyl-amino)-6,7,8,9-tetrahydro-5H-benzocyclohepten-6-ol as colourless crystals (3 g, 52% from 5,6,8,9-tetrahydro-benzocyclohepten-7-one).

Melting point: 148-150° C.

### Preparation 6: 7-(tert-butoxycarbonyl-amino)-5,7,8,9-tetrahydro-benzocyclohepten-6-one

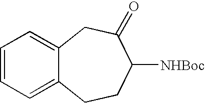

This compound corresponds to a derivative of the formula (III).

To a solution of 7-(tert-butoxycarbonyl-amino)-6,7,8,9-tetrahydro-5H-benzocyclohepten-6-ol (preparation 4) (1 g, 3.6 mmoles) in dichloromethane (20 mL), DMP (Dess-Martin periodinane) (2.3 g, 5.4 mmoles) is added and the mixture is stirred at room temperature under argon for 3 hours. The reaction mixture is diluted with ethyl acetate (50 mL), sodium thiosulphate pentahydrate (6.7 g, 27 mmoles, 5 eq.) and a 1 N aqueous sodium hydrogencarbonate solution are added, and stirred at room temperature for 1 hour. The organic phase is washed several times with a 1N aqueous sodium hydrogencarbonate solution and brine, and dried on magnesium sulphate. The solvent is evaporated off, and the resulting solid is washed with isopropyl ether (3 times) to obtain colourless crystals of 7-(tert-butoxycarbonyl-amino)-5,7,8,9-tetrahydro-benzocyclohepten-6-one (765 mg, 77%).

Melting point: 150-152° C.

¹HNMR (CDCl₃): 7.18 (m, 4 Har); 5.43 (d, NH); 4.55 (dt, H—C(7)); 3.95 (d, Ha-C(5)); 3.60 (d, Hb-C(5)); 3.03 (ddd, Ha-C(9)); 2.89 (ddd, Hb-C(9)); 2.63 (dddd, Ha-C(8)); 1.46 (m, Hb-C(8)); 1.43 (s, tBu); J(5a,5b)=14.6 Hz, J(7,NH)=ca 7.0 Hz, J(7, 8a)=7.0 Hz, J(7,8b)=11.3 Hz, J(8a,8b)=12.8 Hz, J(8a,9a)=9.0 Hz, J(8a,9b)=3.4 Hz, J(8b,9a)=3.4 Hz, J(8b,9b)=9.0 Hz, J(9a,9b)=14.6 Hz.

### Preparation 7: 7-(benzyloxycarbonyl-amino)-5,7,8,9-tetrahydro-benzocyclohepten-6-one

The synthesis described according to preparation 6 is repeated using 7-(benzyloxycarbonyl-amino)-6,7,8,9-tetrahydro-5H-benzocyclohepten-6-ol synthesized according to preparation 5 (2.71 g, 8.7 mmoles) and DMP (5.2 g, 12.2 mmoles) to obtain colourless crystals of 7-(benzyloxycarbonyl-amino)-5,7,8,9-tetrahydro-benzocyclohepten-6-one (2.48 g, 92%).

Melting point: 119-121° C.

¹HNMR (CDCl₃): 7.35-7.30 (m, 5 Har); 7.22-7.15 (m, 4 Har); 5.71 (d, NH); 5.08 (s, OBn); 4.61 (m, H—C(7)); 3.97 (d, Ha-C(5)); 3.61 (d, Hb-C(5)); 3.06 (ddd, Ha-C(9)); 2.90 (ddd, Hb-C(9)); 2.68 (m, Ha-(C8)); 1.50 (m, Hb-C(8)). J(5a,5b)=14.2 Hz, J(7,NH)=ca. 6.0 Hz, J(7,8a)=7.6 Hz, J(7,8b)=11.6 Hz, J (8a, 9a)=9.2 Hz, J (8a, 9b)=3.2 Hz, J (8a, 8b)=11.4 Hz, J(8b,9a)=3.0 Hz, J(8b,9b)=8.8 Hz, J(9a,9b)=14.6 Hz.

### EXAMPLES

### Example 1

### 7-amino-5,7,8,9-tetrahydro-benzocyclohepten-6-one oxime hydrochloride

To a solution of 5,6,8,9-tetrahydro-benzocyclohepten-7-one (preparation 3, 1.0 g, 6.24 mmoles) in 2N dried hydrochloric acid in methanol (13 mL), n-butyl nitrite (1.1 mL, 9.3 mmoles) is added under argon at 0° C. The reaction mixture is stirred at 0° C. for 45 minutes, and is hydrolysed with 1N aqueous sodium hydrogencarbonate. After extraction with ethyl ether, the organic phase is washed with 1N aqueous sodium hydrogencarbonate, and then with water and dried on magnesium sulphate. After the solvents are evaporated off, the resulting solid is washed with isopropanol to obtain 7,7-dimethoxy-5,7,8,9-tetrahydro-benzocyclohepten-6-one oxime (903 mg, 62%).

A solution of 7,7-dimethoxy-5,7,8,9-tetrahydro-benzocyclohepten-6-one oxime (1.0 g, 4.26 mmoles), 6N aqueous hydrochloric acid (18 mL) and ethyl ether (18 mL) is stirred under argon at 0° C. for 15 minutes. The reaction mixture is extracted with ethyl ether, the organic solution is washed with 1N aqueous sodium hydrogencarbonate and then with water and dried on magnesium sulphate. The solvent is evaporated off and the residue is purified by chromatography (7/3 to 5/5 cyclohexane/ethyl acetate) to obtain 8,9-dihydro-5H-benzocycloheptene-6,7-dione 6-oxime (694 mg, 86%).

A solution of 8,9-dihydro-5H-benzocycloheptene-6,7-dione 6-oxime (2.32 g, 12.3 mmoles) and benzylamine (1.35 mL, 12.4 mmoles) in pyridine (7 mL) is stirred under argon at room temperature for 6 hours. The solution is diluted with methanol (7 mL), sodium borohydride (0.55 g, 14.5 mmoles) is added and the reaction mixture is stirred at room temperature for 1 hour. After dilution with ethyl ether, the organic phase is washed with 1N aqueous sodium hydrogencarbonate and then with water and dried on magnesium sulphate. After the solvents are evaporated off, the resulting solid is washed with diisopropyl ether to obtain off white crystals of 7-benzylamino-5,7,8,9-tetrahydro-benzocyclohepten-6-one oxime (3.18 g, 93%), corresponding to a compound of the formula III, with X being the NOH radical.

Melting point: 144-146° C. (iPr₂O)

¹HNMR (CDCl₂): 7.32 (m, 6 Har); 7.14 (m, 3 Har); 3.95 (d, Ha-C(5)); 3.81 (d, Ha-C(NBn)); 3.72 (d, Hb-C(5)); 3.67 (d, Hb-C(NBn)); 3.50 (dd, H—C(7)); 3.14 (m, Ha-C(9)); 2.65 (m, Hb-C(9)); 2.07 (m, Ha-C(8)); 1.85 (m, Hb-C(8)); J(NCH₂Ph)=12.9 Hz, J(5a,5b)=14.3 Hz, J(7,8a)=4.8 Hz, J(7,8b)=6.4 Hz, J(8a,8b)=13.6 Hz, J(8a,9a)=10.8 Hz, J(8a,9b)=2.9 Hz, J(8b,9a)=2.6 Hz, J(8b,9b)=7.2 Hz, J(9a,9b)=14.4 Hz.

The corresponding hydrochloride is obtained by deprotecting the amine function according to the method below.

The resulting 7-benzylamino-5,7,8,9-tetrahydrobenzocyclohepten-6-one oxime (100 mg, 0.356 mmole) is hydrogenized in ethanol (3 mL) and 1N aqueous hydrochloric acid (357 μL, 0.357 mmole) in the presence of 5% palladium on carbon (7 mg) under hydrogen (1 atm) at room temperature for 13 hours. The catalyst is removed by centrifugation and the solvent is evaporated off. The resulting compound is crystallized in 2-propanol/diethyl ether to obtain beige crystals of 7-amino-5,7,8,9-tetrahydro-benzocyclohepten-6-one oxime hydrochloride (60 mg, 75%).

Melting point: 270-280° C.

¹HNMR (CD₃OD): 7.26-7.18 (m, 4 Har); 4.23 (d, Ha-C(5)); 4.01 (dd, H—C (7)); 3.42 (d, Hb-C(5)); 3.04 (ddd, Ha-C(9)); 2.93 (ddd, Hb-C (9)); 2.41 (m, Ha-C(8)); 1.66 (m, Hb-C(8)); J(5a,5b)=15.2 Hz, J(7,8a)=5.4 Hz, J(7,8b)=11.6 Hz, J(8a,8b)=12.6 Hz, J(8a,9a)=3.4 Hz, J(8a,9b)=8.6 Hz, J(8b,9a)=9.0 Hz, J(8b,9b)=3.4 Hz, J(9a,9b)=14.6 Hz.

### Example 2

### 7-amino-5,7,8,9-tetrahydro-benzocyclohepten-6-one hydrochloride

This step corresponds to deprotecting the amine function of the derivatives of the formula (III) or of the formula (IV).

A mixture of 7-(tert-butoxycarbonyl-amino)-5,7,8,9-tetrahydro-benzocyclohepten-6-one (compound of the formula (III), preparation 6) (765 mg, 2.78 mmoles) and 2.2M hydrogen chloride in ethyl ether (5 mL) is stirred in dioxane (5 mL) at room temperature under argon for 72 hours. The resulting solid is filtered and recrystallized in a 2-propanol/diethyl ether mixture to obtain colourless crystals of 7-amino-5,7,8,9-tetrahydro-benzocyclohepten-6-one hydrochloride (500 mg, 85%).

Melting point: 230-240° C. (dec.)

¹HNMR (CDCl₃): 7.25 (m, 4 Har); 4.38 (dd, H—C(7)); 4.22 (d, Ha-C(5)); 3.63 (d, Hb-C(5)); 3.26 (ddd, Ha-C(9)); 3.04 (ddd, Hb-C(9)); 2.54 (m, Ha-C(8)); 1.69 (m, Hb-C(8)); J(5a,5b)=13.8 Hz, J(7,8a)=6.8 Hz, J(7,8b)=12.1 Hz, J(8a,8b)=12.8 Hz, J(8a,9a)=2.8 Hz, J(8a,9b)=8.2 Hz, J(8b,9a)=10.0 Hz, J(8b,9b)=3.0 Hz, J(9a,9b)=14.8 Hz.

### Example 3

### 7-amino-5-fluoro-5,7,8,9-tetrahydro-benzocyclohepten-6-one hydrochloride

A lithium hexamethyldisilazane solution (1.6 mmoles) is prepared from 1.6M n-butyl lithium (1 mL, 1.6 mmoles) in hexane and hexamethyldisilazane (340 μL, 1.6 mmoles) stirred under argon at −78° C. for 15 minutes.

To this solution, a solution of 7-(tert-butoxycarbonyl-amino)-5,7,8,9-tetrahydro-benzocyclohepten-6-one (preparation 6) (200 mg, 0.73 mmole) and hexamethylphosphoramide (380 μL, 2.18 mmoles) in anhydrous tetrahydrofuran (4 mL) is added dropwise at −78° C. The reaction mixture is stirred under argon at −78° C. for 0.5 hour, and then tert-butyldimethylsilyl chloride (263 mg, 1.74 mmoles) is added and the resulting reaction mixture is stirred at −78° C. for further 15 minutes, diluted with diethyl ether and hydrolyzed by 2M aqueous ammonium chloride, washed with brine, and dried on magnesium sulphate. The solvents are evaporated off and the silylated enol ether is used as such without being further purified.

To a silylated enol ether solution in a mixture of acetonitrile (8 mL) and 1M aqueous sodium hydrogencarbonate solution (2 mL), Selectfluor (309 mg, 0.87 moles) is added under argon at 0° C. The reaction mixture is stirred at 0° C. for 0.5 hour, diluted with ethyl acetate and hydrolyzed with 2M aqueous ammonium chloride, washed with brine and dried on magnesium sulphate. After evaporated to dryness, the residue is dissolved in tetrahydrofuran (10 mL) and tetrabutylammonium fluoride (76 mg, 0.29 mmole) is added. The reaction mixture is stirred under argon at 0° C. for 10 minutes, diluted with ethyl acetate, hydrolyzed with 2M aqueous ammonium chloride, washed with brine and then dried on magnesium sulphate. The solvent is evaporated off and the residue is purified by chromatography (8/2 cyclohexane/ethyl acetate) to obtain colourless crystals of 7-(tert-butoxycarbonyl-amino)-5-fluoro-5,7,8,9-tetrahydro-benzocyclohepten-6-one (110 mg, 52%).

Melting point: 127-129° C.

¹HNMR (CDCl₃): 7.30 (m, 4 Har); 5.57 (d, H—C(5)); 5.50 (d, NH); 5.27 (m, H—C (7)); 3.50 (t, Ha-C(9)); 2.77 (m, Hb-C(9)); 2.67 (m, Ha-C(8)); 1.55 (m, Hb-C(8)); 1.46 (s, tBu); J(5,F)=50.0 Hz, J(7,NH)=6.0 Hz, J(7,8a)=12.0 Hz, J(7,8b)=6.0 Hz, J(8a,8b)=13.1 Hz, J(8a,9b)=J(8b,9a)=6.0 Hz, J(9a,9b)=14.0 Hz.

The corresponding hydrochloride is obtained by deprotecting the amine function according to example 2.

### Example 4

### 7-amino-5-benzylidene-5,7,8,9-tetrahydro-benzocyclohepten-6-one hydrochloride

To a solution of lithium hexamethyldisilazane (0.8 mmoles) (prepared from 1.6 M n-butyl lithium (0.5 mL, 0.8 mmoles, 2.2 eq.) in hexane and hexamethyldisilazane (170 μL, 0.8 mmoles, 2.2 eq.) stirred under argon at −78° C. for 15 minutes), a solution of 7-(tert-butoxycarbonyl-amino)-5,7,8,9-tetrahydro-benzocyclohepten-6-one (preparation 6) (100 mg, 0.36 mmole) and hexamethylphosphoramide (190 μL, 1.1 mmoles, 3 eq.) is added dropwise in anhydrous tetrahydrofuran (3 mL) at −78° C. The reaction mixture is stirred under argon at −78° C. for 20 minutes, and then a benzaldehyde solution (74 μL, 0.73 mmole, 2 eq.) is added in tetrahydrofuran (2 mL) and the resulting reaction mixture is stirred at −78° C. for 1 more hour and then for 2.5 hours at room temperature. The reaction mixture is diluted with ethyl acetate and hydrolyzed with 2M aqueous ammonium chloride, washed with brine, and then dried on magnesium sulphate. The solvents are evaporated off and the residue is purified by chromatography (7/3 cyclohexane/ethyl acetate) to obtain colourless crystals of 5-benzylidene-7-(tert-butoxycarbonyl-amino)-5,7,8,9-tetrahydro-benzocyclohepten-6-one (70 mg, 53%).

Melting point: 156-158° C.

¹HNMR (CDCl₃): 7.95 (s, H—C (1′)); 7.29-7.09 (m, 9

Har); 5.49 (dl, NH); 4.45 (ddd, H—C(7)); 2.99 (ddd, Ha-C(9)); 2.76 (ddd, Hb-C(9)); 2.56 (dddd, Ha-C(8)); 1.74 (m, Hb-C(8)); 1.41 (s, tBu); J(7,NH)=ca 7.0 Hz, J(7,8a)=8.6 Hz, J(7,8b)=10.4 Hz, J(8a,8b)=12.6 Hz, J(8a,9a)=13.0 Hz, J(8a,9b)=7.4 Hz, J(8b,9a)=7.6 Hz, J(8b, 9b)=1.2 Hz, J(9a,9b)=13.8 Hz.

The corresponding hydrochloride is obtained by deprotecting the amine function according to example 2 using 20 mg (55 μmoles) of 5-benzylidene-7-(tert-butoxycarbonyl-amino)-5,7,8,9-tetrahydro-benzocyclohepten-6-one and 2.2M hydrogen chloride in diethyl ether (0.5 mL) in dioxane (0.5 mL). 13 mg (79%) of 7-amino-5-benzylidene-5,7,8,9-tetrahydro-benzocyclohepten-6-one hydrochloride is obtained.

¹HNMR (CD₃OD): 8.05 (s, H—C (1′)); 7.46-7.39 (m, 2 Har); 7.32-7.13 (m, 7 Har); 4.00 (dd, H—C(7)); 3.09 (ddd, Ha-C(9)); 2.96 (ddd, Hb-C(9)); 2.45 (dddd, Ha-C(8)); 2.08 (dddd, Hb-C(8)); J(7,8a)=8.2 Hz, J(7,8b)=11.0 Hz, J(8a,8b)=13.0 Hz, J(8a,9a)=12.6 Hz, J(8a,9b)=7.4 Hz, J(8b,9a)=7.6 Hz, J(8b,9b)=1.2 Hz, J(9a,9b)=14.2 Hz.

### Example 5

### 7-amino-5-benzyl-5,7,8,9-tetrahydro-benzocyclohepten-6-one hydrochloride

The 5-benzylidene-7-(tert-butoxycarbonylamino)-5,7,8,9-tetrahydro-benzocyclohepten-6-one prepared in example 4 is used and hydrogenated in the presence of 5% palladium on carbon to obtain 5-benzyl-7-(tert-butoxycarbonyl-amino)-5,7,8,9-tetrahydro-benzocyclohepten-6-one.

The corresponding hydrochloride is obtained by deprotecting the amine function according to example 2 using 25 mg (68 μmoles) of 5-benzyl-7-(tert-butoxycarbonyl-amino)-5,7,8,9-tetrahydro-benzocyclohepten6-one and 2.2M hydrogen chloride in diethyl ether (0.5 mL) in dioxane (0.5 mL). 18 mg (86%) of colourless crystals of 7-amino-5-benzyl-5,7,8,9-tetrahydro-benzocyclohepten-6-one hydrochloride are obtained.

Melting point: 220-222° C.

¹HNMR (CD₃OD): 7.25-7.16 (m, 9 Har); 4.56 (dd, H—C (5)); 4.24 (dd, H—C (7)); 3.62 (dd, Ha-C (1′)); 3.26 (ddd, Ha-C(9)); 3.22 (dd, Hb-C(1′)); 2.85 (ddd, Hb-C(9)); 2.55 (dddd, Ha-C(8)); 1.65 (dddd, Hb-C(8)); J(1′a,1′b)=13.8 Hz, J(1′a,5)=8.6 Hz, J(1'b,5)=5.8 Hz, J(7,8a)=7.4 Hz, J(7,8b)=11.4 Hz, J(8a,8b)=12.8 Hz, J(8a,9a)=2.8 Hz, J(8a,9b)=8.6 Hz, J(8b,9a)=9.8 Hz, J(8b,9b)=3.0 Hz, J(9a, 9b)=14.6 Hz.

### Example 6

### 5-phenylpropyl-7-amino-5,7,8,9-tetrahydro-benzocyclohepten-6-one hydrochloride

A mixture of 7-(benzyloxycarbonyl-amino)-5,7,8,9-tetrahydro-benzocyclohepten-6-one (preparation 7) (300 mg, 0.97 mmole), 36% aqueous formaldehyde (225 μL, 2.91 mmoles), and pyrrolidine (50 μL) is heated in acetic acid (10 mL) at 110° C. under argon for 5 hours. The reaction mixture is diluted with ethyl acetate, washed with 1M aqueous sodium hydrogencarbonate and then with brine, and dried on magnesium sulphate. The solvent is evaporated off to obtain a yellowish resin of 5-methylene-7-(benzyloxycarbonyl-amino)-5,7,8,9-tetrahydro-benzocyclohepten-6-one.

Then, to a copper(I) bromide-dimethyl sulphide complex suspension (440 mg, 2.13 mmoles) in anhydrous tetrahydrofuran (20 mL), a solution of phenylethyl magnesium bromide (3.1 mL, 1.3 M in diethyl ether, 4.27 mmoles) is added dropwise under argon at −50° C. The reaction mixture is stirred at −50° C. for 45 minutes, and then a solution of 5-methylene-7-(benzyloxycarbonyl-amino)-5,7,8,9-tetrahydro-benzocyclohepten-6-one (0.97 mmole) is added dropwise in aqueous tetrahydrofuran (10 mL). The reaction mixture is stirred at −40° C. for 2 hours, hydrolyzed with 2M aqueous ammonium chloride, extracted with ethyl acetate, washed with brine, and then dried on magnesium sulphate. The solvent is evaporated off and the residue is purified by chromatography (9/1 and then 8/2 cyclohexane/ethyl acetate) to obtain colourless crystals of 5-phenylpropyl-7-(benzyloxycarbonyl-amino)-5,7,8,9-tetrahydro-benzocyclohepten-6-one (182 mg, 43%).

Melting point: 106-108° C.

¹HNMR (CDCl₃): 7.34-7.11 (m, 14 Har); 5.67 (d,NH); 5.08 (s, OCH₂Ph); 4.58 (m, H—C(7)); 3.91 (m, H—C(5)); 3.06 (m, Ha-C(9)); 2.83 (m, Hb-C(9)); 2.70-2.60 (m, Ha-C(8), 2H—C(3′)); 2.37 (m, Ha-C(1′)), 1.82 (m, Hb-C(1′)); 1.63 (m, 2H—C(2′)); 1.43 (m, Hb-C(8)).

The corresponding hydrochloride is obtained by deprotecting the amine function according to the method below.

The resulting 5-phenylpropyl-7-(benzyloxycarbonyl-amino)-5,7,8,9-tetrahydro-benzocyclohepten-6-one (139 mg, 0.325 mmole) is hydrogenolysed in dioxane (20 mL) and 1N aqueous hydrochloric acid (0.36 mL, 0.36 mmole) in the presence of 5% palladium on carbon (7 mg) under hydrogen (1 atm) at 40° C. for 24 hours. The catalyst is removed by centrifugation and the solvent is evaporated off. The resulting compound is recrystallized in 2-propanol/diethyl ether to obtain colourless crystals of 5-phenylpropyl-7-amino-5,7,8, 9-tetrahydro-benzocyclohepten-6-one hydrochloride (80 mg, 75%).

Melting point: 156-158° C.

¹HNMR (CD₃OD): 7.27-7.15 (m, 9 Har); 4.32 (dd, H—C (7)); 4.19 (dd,H—C(5)); 3.28 (m, Ha-C(9)); 2.95 (ddd, Hb-C(9)); 2.70 (m, 2H—C(3′)); 2.55 (m, Ha-C(8)); 2.34 (m, Ha-C(1′)); 1.88 (m, Hb-C(1′)); 1.67 (tt, 2H—C(2′)); 1.61 (m, Hb-C(8)); J(1a′,1b′)=13.2 Hz, J(1a′,2′)=8.0 Hz, J(1a′,5)=8.2 Hz, J(1b′,2′)=8.0 Hz, J(1b′,5)=6.0 Hz, J(2′,3′)=7.2 Hz, J(7,8a)=7.2 Hz, J(7,8b)=11.8 Hz, J(8a,8b)=12.6 Hz, J(8a,9a)=2.6 Hz, J(8a,9b)=7.8 Hz, J(8b,9b)=2.6 Hz, J(9a,9b)=14.6 Hz.

By substituting benzyl magnesium bromide for phenylethyl magnesium bromide, 5-phenylthyl-7-amino-5,7,8,9-tetrahydro-benzocyclohepten-6-one hydrochloride is obtained in the same way.

### Example 7

### 7-amino-4-phenyl-5,7,8,9-tetrahydro-benzocyclohepten-6-one hydrochloride

A mixture of 7-(tert-butoxycarbonylamino)-4-bromo-5,7,8,9-tetrahydro-benzocyclohepten-6-one (90 mg, 0.25 mmole), phenylboronic acid (35 mg, 0.28 mmole), cesium fluoride (86 mg, 0.56 mmole) and tetrakistriphenylphosphine palladium (30 mg, 0.025 mmole) is heated in anhydrous 1,2-dimethoxylethane (3 mL) under argon at 85° C. for 5 hours. The reaction mixture is diluted with ethyl acetate, washed with brine and dried on magnesium sulphate. The solvent is evaporated off and the residue is purified by chromatography (8/2 cyclohexane/ethyl acetate) to obtain colourless crystals of 7-(tert-butoxycarbonyl-amino)-4-phenyl-5,7,8,9-tetrahydro-benzocyclohepten-6-one (70 mg, 78%).

Melting point: 173-174° C.

¹HNMR (CDCl₃): 7.45-7.17 (m, 8 Har); 5.44 (d, NH); 4.55 (ddd,H—C(7)); 3.79 (d, Ha-C(5)); 3.72 (d, Hb-C(5)); 3.07 (m, Ha-C(9)); 2.96 (m, Hb-C(9)); 2.67 (m, Ha-C(8)); 1.54 (m, Hb-C(8)); 1.42 (s, tBu); J(7,NH)=ca 6.7 Hz, J(5a,5b)=15.5 Hz, J(7,8a)=7.8 Hz, J(7,8b)=11.8 Hz, J(8a,9a)=3.6 Hz, J(8a,9b)=9.5 Hz, J(8b,9a)=8.4 Hz, J(8a,9b)=3.9 Hz, J(9a,9b)=15.0 Hz.

The corresponding hydrochloride is obtained by deprotecting the amine function according to example 2 using 50 mg (0.14 mmole) of 7-(tert-butoxycarbonyl-amino)-4-phenyl-5,7,8,9-tetrahydro-benzocyclohepten-6-one and 2.2M hydrogen chloride in diethyl ether (0.5 mL) in dioxane (0.5 mL). 30 mg (73%) of colourless crystals of 7-amino-4-phenyl-5,7,8,9-tetrahydro-benzocyclohepten-6-one hydrochloride are obtained.

Melting point: 222° C.

¹HNMR (CD₃OD): 7.41 (m, 5 Har); 7.28 (m, 2 Har); 7.20 (m, 1 Har); 4.39 (dd, H—C(7)); 3.99 (d, Ha-C(5)); 3.77 (d, Hb-C(5)); 3.32 (m, Ha-C(9)); 3.13 (ddd, Hb-C(9)); 2.60 (m, Ha-C(8)); 1.76 (m, Hb-C(8)); J(5a,5b)=14.4 Hz, J(7,8a)=7.2 Hz, J(7,8b)=12.4 Hz, J(8a,8b)=13.2 Hz, J(8a,9a)=2.7 Hz, J(8a,9b)=8.5 Hz, J(8b,9a)=10.6 Hz, J(8b,9b)=3.0 Hz, J(9a,9b)=15.3 Hz.

### Example 8

### 5-benzylsulphanyl-7-amino-5,7,8,9-tetrahydro-benzocyclohepten-6-one hydrochloride

To a solution of 5,6,8,9-tetrahydro-benzocyclohepten-7-one (preparation 3) (3.07 g, 19 mmoles), triethylamine (3.7 mL, 27 mmoles) in anhydrous toluene (20 mL), triethylsilyl trifluoromethanesulphonate (4.9 mL, 23 mmoles) is added dropwise at room temperature under argon. The reaction mixture is heated at 90° C. for 2 hours and diluted with cyclohexane, and then washed with brine. The organic phase is dried on magnesium sulphate and evaporated to obtain a silylated enol ether used without being further purified.

This silylated enol ether (19 mmoles), palladium (II) acetate (430 mg, 1.9 mmoles) in anhydrous dimethylsulphoxide (25 mL) are stirred at room temperature under oxygen (1 atm) for 20 hours. The reaction mixture is diluted with diethyl ether, washed with brine and then dried on magnesium sulphate. The solvent is evaporated off and the residue is distilled off at 82° C. under 0.05 Torr to obtain 5,6-dihydro-benzocyclohepten-7-one (2.59 g, 85%).

Then, a solution of 5,6-dihydro-benzocyclohepten-7-one (0.5 g, 3.16 mmoles), titanium (IV) isopropoxide (1.9 mL, 6.33 mmoles) and ammoniac saturated in ethanol (10 mL) is stirred under argon overnight. Sodium borohydride (132 mg, 3.48 mmoles) is then added, and the reaction mixture is stirred at room temperature for 1 further hour. The solvents are evaporated off and the residue is diluted with ethyl acetate, and 1N aqueous ammonium hydroxide (20 mL) is added. The resulting inorganic precipitate is filtered and washed with a mixture of 1/1 ethyl acetate and 1N aqueous ammonium hydroxide (3×20 mL). The organic phase is separated and the remaining aqueous phase is extracted with ethyl acetate (3×20 mL). The combined organic extracts are dried on magnesium sulphate and concentrated to dryness to obtain ethylenic amine.

A mixture of this ethylenic amine, di-tert-butyl dicarbonate (1.4 g, 6.33 mmoles), and sodium carbonate (370 mg, 3.48 mmoles) is stirred in methanol (6 mL) under argon for 3 hours. The solids are filtered off, the solvent is evaporated off and the consulting solid is washed with water (3×20 mL) and cold isopropyl ether (3×20 mL) to obtain colourless crystals of 7-(tert-butoxycarbonyl-amino)-6,7-dihydro-5H-benzocycloheptene (625 mg, 76%) corresponding to a derivative of the formula (V):

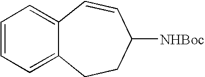

Melting point: 146-148° C.

¹HNMR (CDCl₃): 7.18-7.11 (m, 4 Har); 6.44 (dd, H—C(9)); 5.76 (dd,H—C(8)); 4.69 (d, NH); 4.49 (s, H—C(7)); 2.84 (m, Ha-C(5)); 2.73 (m, Hb-C(5)); 2.04 (m, 2H—C(6)); 1.46 (s, tBu); J(7,8)=4.0 Hz, J(7,9)=1.9 Hz, J(8,9)=12.3 Hz.

To a solution of 7-(tert-butoxycarbonyl-amino)-6,7-dihydro-5H-benzocycloheptene (400 mg, 1.54 mmole) in anhydrous dichloromethane (20 mL), 3-chloroperoxybenzoic acid (610 mg, 2.46 mmoles) is added portionwise under argon at 0° C. The reaction mixture is stirred at room temperature overnight, and diluted with ethyl acetate (10 mL), sodium thiosulphate pentahydrate (3.05 g, 12.3 mmoles) and 1N aqueous sodium hydrogencarbonate are added and the whole is stirred at room temperature for 1 hour. The organic solution is washed consecutively with 1N aqueous sodium hydrogencarbonate and brine, and dried on magnesium sulphate. The solvent is evaporated off, and the resulting solid is recrystallized in 2-propanol to obtain colourless crystals of 7-(tert-butoxycarbonyl-amino)-5,6-epoxy-6,7,8,9-tetrahydro-5H-benzocycloheptene (390 mg, 90%).

Melting point: 170-172° C.

¹HNMR (CDCl₃): 7.50 (m, 1 Har); 7.23 (m, 2 Har); 7.08 (m, 1 Har); 4.98 (d, NH); 4.36 (m, H—C (7)); 3.99 (d,H—C (5)); 3.68 (d, H—C (6)); 2.84 (dd, Ha-C(9>>; 2.63 (dd, Hb-C(9)); 1.95 (m, Ha-C(8)); 1.69 (m, Hb-C(8)); 1.47 (s, tBu); J(5,6)=4.2 Hz, J(6,7)=2.4 Hz, J(NH, 7)=9.0 Hz, J(7,8a)=4.4 Hz, J(7,8b)=10.6 Hz, J(8a,8b)=13.4 Hz, J(8a,9a)=8.8 Hz, J(8b,9b)=10.4 Hz, J(9a,9b)=15.4 Hz.

A solution of 7-(tert-butoxycarbonyl-amino)-5,6-epoxy-6,7,8,9-tetrahydro-5H-benzocycloheptene (52 mg, 0.19 mmole), triethylamine (63 μL, 0.45 mmole) and benzenethiol (30 μL, 0.23 mmole) is stirred in ethanol (1 mL) under argon at room temperature overnight. The reaction mixture is diluted with water and the resulting precipitate is filtered, washed with isopropyl ether (3 times) to obtain the derivative corresponding to the formula (VI) 5-benzylsulphanyl-7-(tert-butoxycarbonyl-amino)-6,7,8,9-tetrahydro-5H-benzocyclohepten-6-ol (60 mg, 80%).

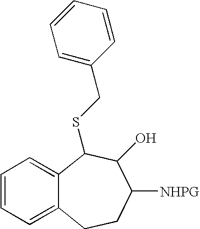

¹HNMR (CDCl₃): 7.32-7.15 (m, 6 Har); 7.15 (t, J=7.3 Hz, 2 Har); 6.98 (d, J=7.3 Hz, 1 Har); 4.99 (d, J=6.0 Hz, NH); 4.22 (m, H—C(7)); 4.09 (m, H—C(6)); 3.94 (d, J=6.0 Hz, H—C (5)); 3.71 (d, J=13.8 Hz, Ha-(SBn)); 3.56 (d, J=13.8 Hz, Hb-(SBn)); 3.38 (t, J=13.8 Hz, Ha-C(9)); 2.66 (m, Hb-C(9)); 2.00 (m, Ha-C(8)); 1.46 (m, Hb-C(8)); 1.45 (s, tBu).

To obtain the 5-benzylsulphanyl-7-(tert-butoxycarbonyl-amino)-5,7,8,9-tetrahydrobenzocyclohepten-6-one (40 mg), the synthesis described according to preparation 6 is repeated using 5-benzylsulphanyl-7-(tert-butoxycarbonyl-amino)-6,7,8,9-tetrahydro-5H-benzocyclohepten-6-ol (40 mg, 0.1 mmole) and Dess-Martin periodinane (47 mg, 0.11 mmole) in 5 mL of CH₂Cl₂.

¹HNMR (CDCl₃): 7.30-7.03 (m, 9 Har); 5.34 (m, NH, H—C(7); 4.42 (s, H—C(5)); 3.72 (s, SCH₂Ph); 3.53 (m, Ha-C(9)); 2.74 (m, Hb-C(9)); 2.53 (m, Ha-C(8)); 1.43 (m, Hb-C(8)); 1.42 (s, tBu).

Deprotecting the amine is carried out the same way as in example 2 using 40 mg (0.1 mmole) of 5-benzylsulphanyl-7-(tert-butoxycarbonyl-amino)-5,7,8,9-tetrahydrobenzocyclohepten-6-one, 2.2M hydrogen chloride in diethyl ether (0.5 mL) in dioxane (0.5 mL), to obtain colourless crystals of 5-benzylsulphanyl-7-amino-5,7,8,9-tetrahydrobenzocyclohepten-6-one hydrochloride (20 mg, 66%).

Melting point: 176-180° C.

¹HNMR (CDCl₃): 7.36-7.09 (m, 9 Har); 5.18 (dd, H—C(7)); 4.60 (s, H—C(5)); 3.86 (d, Ha-C(SBn)); 3.79 (d, Hb-C(SBn)); 3.64 (dd, Ha-C(9)); 2.98 (ddd, Hb-C(9)); 2.54 (m, Ha-C(8)); 1.76 (m, Hb-C(8)); J(SBn)=13.7 Hz, J(7,8a)=6.0 Hz, J(7,8b)=12.5 Hz, J(8a,8b)=13.5 Hz, J(8a,9a)=2.0 Hz, J(8a,9b)=8.0 Hz, J(8b,9a)=12.0 Hz, J(8b,9b)=2.0 Hz, J(9a,9b)=15.6 Hz.

### Example 9

### 7-amino-1-bromo-5,7,8,9-tetrahydro-benzocyclohepten-6-one hydrochloride

The synthesis according to preparations 2, 3, 4, and 6 are repeated but using at the beginning the compound I-bromo-2,3-bis-bromomethyl-benzene (preparation 1) instead of dibromo-o-xylene. 7-(tert-butoxycarbonyl-amino)-1-bromo-5,7,8,9-tetrahydro-benzocyclohepten-6-one is obtained.

Deprotecting the amine is carried out the same way as in example 2 using 25 mg (71 μmoles) of 7-(tert-butoxycarbonyl-amino)-1-bromo-5,7,8,9-tetrahydro-benzocyclohepten-6-one, 2.2M hydrogen chloride in diethyl ether (0.5 mL) in dioxane (0.5 mL), to obtain colourless crystals of 7-amino-1-bromo-5,7,8,9-tetrahydro-benzocyclohepten-6-one hydrochloride (15 mg, 74%).

Melting point: 117-118° C.

¹HNMR (CD₃OD): 7.57 (d, J=8.2 Hz, 1 Har); 7.25 (d, J=7.7 Hz, 1 Har); 7.14 (t, J=8.0 Hz, 1 Har); 4.29 (dd, H-4.20 (d, Ha-C(5)); 3.79 (d, Hb-C(5)); 3.46 (m, Ha-C(9)); 3.35 (m, Hb-C(9)); 2.54 (m, Ha-C(8)); 1.71 (m, Hb-J(5a,5b)=15.7 Hz, J(7,8a)=7.6 Hz, J(7,8b)=12.1 Hz, J(8a,8b)=13.4 Hz, J(8a,9a)=9.8 Hz, J(8a,9b)=3.6 Hz, J(8b,9a)=3.8 Hz, J(8b,9b)=8.4 Hz, J(9a,9b)=15.4 Hz.

### AP-N Inhibitory Activity

The compounds according to the invention have been tested to show their importance as active substances in therapy.

In particular, they have been tested as AP-N inhibitors.

For this purpose, different known AP-N inhibitory molecules and according to the invention have been tested by measuring their inhibition constant Ki, not only on AP-N but also on cytosolic leucine aminopeptidase (LAPc) and on human recombinant leukotriene A₄ hydrolase (LTA₄H) in order to show the selectivity of the molecules. LTA₄H has an aminopeptidase type activity similar to AP-N and a very close substrate specificity. It is important for the AP-N inhibitory molecules neither to inhibit LTA₄H nor aminopeptidases that contain a co-catalytic unit like LAPc.

The LAPc enzymes from bovine livers and AP-N from porcine livers are marketed by Sigma Chemical Corporation. The AP-N is purified in a soluble form according to the method described by Wacker, H., Lecky, P., Fischer E. H., Stein, E. A. Hely. Chim. Acta, 1971, 54, 473-484.

Tests have been carried out by spectrophotometry with L-leucine para-nitroanilide as a substrate for LAPc (Kₘ=2 mM), AP-N (Kₘ=0.2 mM), and alanine para-nitroanilide for LTA₄H (Kₘ=2 mM). The kinetic studies are carried out at 30° C. and the reactions are started up by adding the enzymes in 1 mL of test medium: LAPc, 5 units in 10 mM Tris-HCl, 0.1 mM ZnCl₂, 5 mM MnCl₂, 1 M KCl, pH=8.0; AP-N, 25 mUnits in 10 mM Tris-HCl, pH=7.5 and LTA₄H, 5 μg in 10 mM Tris-HCl, 0.1 mM KCl, pH=7.5.

The release of para-nitroaniline (ε=10 800 M⁻¹ cm⁻¹) is monitored at 405 nm to determine the initial rates. The inhibition constants Ki are measured by the Dixon method (Segel, H. in Enzyme Kinetics, 1975, pp 109-144).

The results are set out in table III below showing Ki(M) values of different inhibitory molecules assessed for the 3 types of aminopeptidases tested.

All the inhibitory molecules are assessed as an hydrochloride and in racemic mixture.

The inhibitory molecules synthesized according to examples 2, 3, 6, 7, 8, 9 are used.

By way of comparison, molecules known in literature for their AP-N inhibitory activity are used:

2-amino-3-tetralone corresponding to example 10 (comparative)

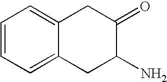

and bestatine corresponding to example 11 (comparative)

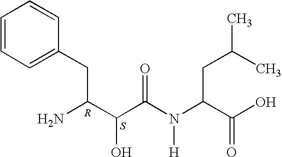

TABLE III

| | Ki (M) | | |
|---|---|---|---|
| | AP-N | LTA₄H | LAPc |
| Molecules | (EC3.4.11.2) | (EC3.3.2.6) | (EC3.4.11.1) |
| EX 2 (inv.) | 1 × 10⁻⁶ | >>10⁻³ | >>10⁻³ |
| EX 3 (inv.) | 3 × 10⁻⁷ | >>10⁻³ | >>10⁻³ |
| EX 6 (inv.) | 1.2 × 10⁻⁵ | >>10⁻³ | >>10⁻³ |
| EX 7 (inv.) | 7 × 10⁻⁹ | >>10⁻³ | 2 × 10⁻⁵ |
| EX 8 (inv.) | 8 × 10⁻⁸ | >>10⁻³ | >>10⁻³ |
| EX 9 (inv.) | 2 × 10⁻⁸ | >>10⁻³ | >>10⁻³ |
| EX 10 | 5 × 10⁻⁷ | >>10⁻³ | 1.2 × 10⁻⁴ |
| (comp.) | | | |
| EX 11 | 3 × 10⁻⁶ | 5 × 10⁻⁷ | 5 × 10⁻¹⁰ |
| (comp.) | | | |

The results from table III show that only the molecules according to the invention have a great AP-N selectivity.

### Stability in Aqueous Solution

For this test, the molecule according to example 2 of the invention and, by way of comparison, 2-amino-3-tetralone (Ex 10) are used as AP-N inhibitors.

The enzymatic tests are carried out at 25° C. in 20 mM Tris-HCl, pH=7.5 with 0.2 mM L-leucine para-nitroanilide as a substrate, in a total reaction volume of 1 mL. The release of para-nitroaniline (ε=10 800 M⁻¹ cm⁻¹) is monitored at 405 nm. The reaction is started up by adding 3 milliunits of AP-N from porcine livers.

Under these experimental conditions, the linear kinetics is monitored for at least 10 hours. The evolution of para-nitroaniline concentration between a sample A without inhibitor, a sample B including 1 μM of 2-amino-3-tetralone (Ex 10) and a sample C including 200 μM of 7-amino-5,7,8,9-tetrahydro-benzocyclohepten-6-one hydrochloride (Ex 2) is compared. For sample B, a 40% inhibition is observed with 2-amino-3-tetralone during the first hour. After 4 hours of test, the enzymatic activity becomes identical again to that of control. On the contrary, for sample C, a 50% inhibition is observed throughout the test, which indicates a stability of the 7-amino-5,7,8,9-tetrahydro-benzocyclohepten-6-one hydrochloride molecule after 10 hours of test.

The same results are obtained with all other tested compounds of the invention.

The compounds according to the invention comprising a 7-carbon atom ring are thus much more stable molecules in aqueous solution than the 2-amino-3-tetralone molecule which comprises a 6-carbon atom ring, already known as a AP-N inhibitor but having the drawback not to be very stable.

1. A compound of the general formula (I): 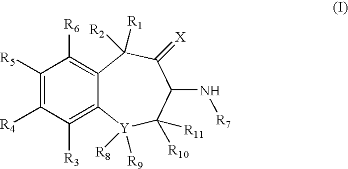
wherein,
R₁ represents an hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical, a (C₁-C₆)alkoxy radical, a (C₁-C₆)aralkyloxy radical, a (C₁-C₆)alkylthio radical, a (C₁-C₆) aralkylthio radical;
R₂ represents an hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical; where R₁ and R₂ can form together an unsubstituted or substituted carbon ring or an unsubstituted or substituted heterocycle; or R₁ can be bonded to the heptene ring through a double bond, R₂ being then absent;
R₃, R₄, R₅ and R₆, the same or different, represent independently of each other a hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a polyfluoro (C₁-C₆)alkyl radical, a (C₁-C₆) aralkyl radical, a (C₁-C₆)heteroaralkyl radical, a (C₁-C₆)alkoxy radical, an aryl or heteroaryl group; R₃ and R₄, R₄ and R₅, R₅ and R₆ independently of each other can form together a methylenedioxy radical joining the adjacent carbon atoms or an unsubstituted or substituted aromatic carbon ring or an unsubstituted or substituted aromatic heterocycle;
R₇ represents a hydrogen atom, a (C₁-C₆)alkyl radical;
X is an oxygen atom, a sulphur atom, an imine radical N—R₁₂, an oxime radical N—O—R₁₃, wherein R₁₂ and R₁₃ represent a hydrogen atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical;
Y is a carbon atom; a nitrogen atom, R₈ or R₉ being then absent; an oxygen atom, a sulphur atom, R₈ and R₉ being then absent;
R₈ and R₁₀, the same or different, represent independently of each other a hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical, a (C₁-C₆)alkoxy radical, a (C₁-C₆)aralkyloxy radical, a (C₁-C₆)alkylthio radical, a (C₁-C₆)aralkylthio radical;
R₉ and R₁₁, the same or different, represent independently of each other a hydrogen, fluorine, chlorine, bromine atom, a (C₁-C₆)alkyl radical, a (C₁-C₆) (cycloalkyl)alkyl radical, a (C₁-C₆) (heterocycloalkyl)alkyl radical, a (C₁-C₆)aralkyl radical, a (C₁-C₆)heteroaralkyl radical, a (C₁-C₆)alkoxy radical, a (C₁-C₆)aralkyloxy radical, a (C₁-C₆)alkylthio radical, a (C₁-C₆)aralkylthio radical, where R₉ and R₁₁ can form together an unsubstituted or substituted carbon ring or an unsubstituted or substituted heterocycle or form a double bond with the two adjacent carbon atoms of the heptene ring;
optical and geometrical isomers thereof and mixtures thereof, as well as inorganic and organic acid addition salts thereof, except for the compound wherein R₄, R₅ and R₆ represent a methoxy radical, R₁, R₂, R₃, R₇, R₈, R₉, R₁₀, R₁₁ represent a hydrogen atom, X represents an oxygen atom and Y represents a carbon atom.

2. The compound according to claim 1, characterised in that R₁ represents a hydrogen atom, a fluorine atom, a (CH₂)ₙPh radical, a S(CH₂)ₙPh radical, n ranging from 1 to 6;
optical and geometrical isomers thereof and mixtures thereof, as well as inorganic and organic acid addition salts thereof.

3. The compound according to claim 1, characterised in that R₂ is a hydrogen atom;
optical and geometrical isomers thereof and mixtures thereof, as well as inorganic and organic acid addition salts thereof.

4. The compound according to claim 1, characterised in that X is an oxygen atom;
optical and geometrical isomers thereof and mixtures thereof, as well as inorganic and organic acid addition salts thereof.

5. The compound according to claim 1, characterised in that Y is a carbon atom;
optical and geometrical isomers thereof and mixtures thereof, as well as inorganic and organic acid addition salts thereof.

6. The compound according to claim 1, characterised in that R₃, R₄, R₅ and R₆, the same or different, represent independently of each other, a hydrogen atom, a bromine atom, a phenyl radical, or R₃ and R₄, R₄ and R₅, R₅ and R₆ independently of each other form together an unsubstituted or substituted aromatic carbon ring;
optical and geometrical isomers thereof and mixtures thereof, as well as inorganic and organic acid addition salts thereof.

7. The compound according to claim 1, characterised in that R₇ is a hydrogen atom;
optical and geometrical isomers thereof and mixtures thereof, as well as inorganic and organic acid addition salts thereof.

8. The compound according to claim 1, characterised in that simultaneously Y is a carbon atom and R₈, R₉, R₁₀ and R₁₁ are hydrogen atoms;
optical and geometrical isomers thereof and mixtures thereof, as well as inorganic and organic acid addition salts thereof.

9. The compound according to claim 1, characterised in that R₂ and R₇ are simultaneously a hydrogen atom, X is an oxygen atom and Y is a carbon atom.

10. The compound according to claim 1, characterised in that R₁ is a hydrogen atom, a fluorine atom, a benzylthio radical, a (C₂)ₙPh radical,
where n=1-5.

11. The compound according to claim 1, characterised in that R₃, R₄, R₅, R₆ are simultaneously a hydrogen atom.

12. The compound according to claim 10, characterised in that R₄ and R₅ are a hydrogen atom, and R₃ and R₆ represent independently of each other a hydrogen atom, a bromine atom, a phenyl radical, with the proviso that R₃ and R₆ are not simultaneously a hydrogen atom.

13. The compound according to claim 10, characterised in that R₃ and R₆ are a hydrogen atom, and R₄ and R₅ represent independently of each other a hydrogen atom, a bromine atom, a phenyl radical, with the proviso that R₄ and R₅ are not simultaneously a hydrogen atom.

14. The compound according to claim 10, characterised in that R₃ and R₄, R₅ and R₆ independently of each other, form together an unsubstituted or substituted aromatic carbon ring, joining the adjacent carbon atoms.

15. The compound according to claim 1, characterised in that it is of the formula (Ia): 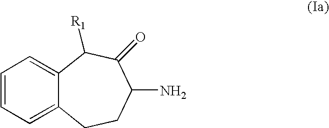
wherein R₁ represents a hydrogen atom, a fluorine atom, the CH₂Ph radical, the (CH₂)₂Ph radical, the (CH₂)₃Ph radical, the (CH₂)₄Ph radical, the (CH₂)₅Ph radical, the S—CH₂Ph radical, the ═CH-Ph radical; as well as inorganic and organic acid addition salts thereof.

16. The compound according to claim 1, characterised in that it is selected from the compounds of the following formulae (Ib) and (Ic); as well as inorganic and organic acid addition salts thereof:

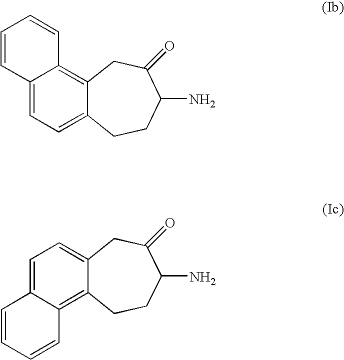

17. The compound according to claim 1, characterised in that it is of the formula (Id): 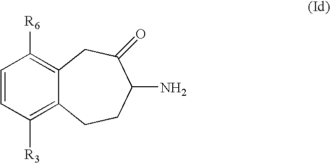
wherein
R₃ is a hydrogen atom and R₆ is a phenyl radical; R₃ is a hydrogen atom and R₆ is a bromine atom; R₃ is a phenyl radical and R₆ is a hydrogen atom; R₃ is a bromine atom and R₆ is a hydrogen atom; R₃ is a phenyl radical and R₆ is a bromine atom; R₃ is a bromine atom and R₆ is a phenyl radical; R₃ and R₆ are a phenyl radical respectively; R₃ and R₆ are a bromine atom respectively;
as well as inorganic and organic acid addition salts thereof.

18. The compound according to claim 1, characterised in that it is of the formula (Ie): 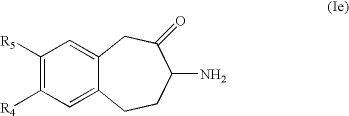
wherein
R₄ is a hydrogen atom and R₅ is a phenyl radical; R₄ is a hydrogen atom and R₅ is a bromine atom, R₄ is a phenyl radical and R₅ is a hydrogen atom, R₄ is a bromine atom and R₅ is a hydrogen atom, R₄ and R₅ are a phenyl radical respectively, R₄ and R₅ are a bromine atom respectively; as well as inorganic and organic acid addition salts thereof.

19. A method for making a compound of formula (I) according to claim 1, wherein R₁ represents a hydrogen atom, a fluorine atom, a (CH₂)ₙPh radical, the ═CH-Ph radical, R₂ is a hydrogen atom or is absent, R₇, R₈, R₉, R₁₀, and R₁₁ are hydrogen atoms; X is an oxygen atom, a NOH radical, Y is a carbon atom, R₃, R₄, R₅, R₆ have the meanings already set out; and salts thereof, characterised in that
1) a —NHPG protected amine function is introduced onto the compound of the general formula (II) 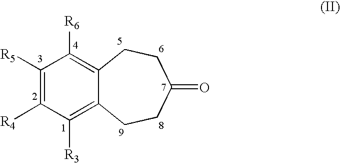
at the 7-position by reacting the ketone function, wherein PG is a protecting group, and at the 6-position, a ketone function is introduced when X is an oxygen atom or a ketone-oxime function is introduced when X is the NOH radical to form a derivative of the general formula (III) 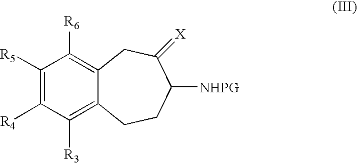
2) when R₁ is not a hydrogen atom, the function corresponding to R₁ is introduced at the 5-position to form a derivative of the general formula (IV) 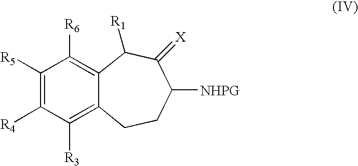
3) the amine NE-PG function is deprotected by cleaving the PG group.

20. The method for making a compound of formula (I) according to claim 1, wherein R₁ represents a S(CH₂)ₙPh radical, R₂ is a hydrogen atom, R₇, R₆, R₉, R₁₀ and R₁₁ are hydrogen atoms; X is an oxygen atom, Y is a carbon atom, R₃, R₄, R₅, R₆ have the meanings already set out;
and salts thereof, characterised in that
1) a double bond is formed onto the compound of the general formula (II) 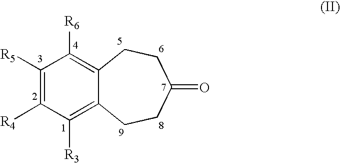
between the 5- and 6-positions
2) a —NHPG protected amine function is introduced at the 7-position by reacting the ketone function, wherein PG is a protecting group to form a derivative of the general formula (V) 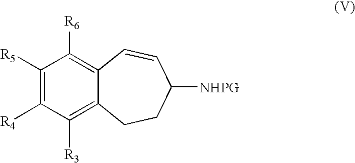
3) the double bond is oxidized to form an epoxide function bonding together the carbon atoms at the 5- and 6-positions
4) the S(CH₂)ₙPh radical is introduced at the 5-position to form a derivative of the general formula (VI) 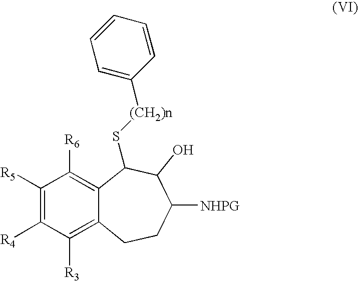
5) the alcohol function of the resulting derivative is oxidized
6) the NH-PG amine function is deprotected by cleaving the PG group.

21. A pharmaceutical composition, characterised in that it contains as an active principle, a compound of the formula (I) according to claim 1, or pharmaceutically acceptable inorganic and organic acid addition salts thereof, with the proviso that the compound wherein R₄, R₅ and R₆ represent a methoxy radical, R₁, R₂, R₃, R₇, R₈, R₉, R₁₀ and R₁₁ represent a hydrogen atom, X represents an oxygen atom and Y represents a carbon atom is not excluded.

22. The pharmaceutical composition according to claim 21, characterised in that the active principle is mixed with at least a pharmaceutically acceptable excipient.

23. A method of making a drug for treating cancer, comprising mixing at least one pharmaceutically acceptable excipient with a compound of the formula (I) according to claim 1, with the proviso that the compound wherein R₄, R₅ and R₆ represent a methoxy radical, R₁, R₂, R₃, R₂, R₈, R₉, R₁₀ and R₁₁ represent a hydrogen atom, X represents an oxygen atom and Y represents a carbon atom is not excluded, for making a drug for treating cancers.

24. A method for treating diseases involving inhibition of metalloproteases, comprising administering to a subject in need thereof, an effective amount of a compound of the formula (I) according to claim 1, with the proviso that the compound wherein R₄, R₅ and R₆ represent a methoxy radical, R₁, R₂, R₃, R₇, R₈, R₉, R₁₀ and R₁₁ represent a hydrogen atom, X represents an oxygen atom and Y represents a carbon atom is not excluded.
